(19) United States
(12) Patent Application Publication
(10) Pub. No.: US 2010/0158854 A1
(43) Pub. Date: Jun. 24, 2010

(54) METHOD FOR TREATMENT OF AIRWAY AND APPARATUS AND KIT FOR USE THEREWITH

(75) Inventor: Nathalie PUISAIS, Argenteuil (FR)

Correspondence Address: MAYER & WILLIAMS PC, 251 NORTH AVENUE WEST, 2ND FLOOR, WESTFIELD, NJ, 07090, US

(73) Assignee: BOSTON SCIENTIFIC SCIMED, INC., Maple Grove, MN (US)

(21) Appl. No.: 12/553,905

(22) Filed: Sep. 3, 2009

Related U.S. Application Data
(60) Provisional application No. 61/139,247, filed on Dec. 19, 2008.

(51) Int. Cl. A61K 31/765; A61F 5/56; A61K 9/00; A61P 1/02

(52) U.S. Cl. 424/78.37; 128/848; 424/435

(57) ABSTRACT

A method of treating an airway in a mammalian body having a mouth, soft palate, uvula, throat and tongue. An implant-forming material is introduced into at least one of the soft palate and uvula for treating at least one of snoring and sleep apnea. An apparatus and kit are provided.

### CROSS-REFERENCE TO RELATED APPLICATIONS

This application claims the benefit of related U.S. Provisional Patent Application Ser. No. 61/139,247 filed Dec. 19, 2008, the entire content of which is hereby incorporated by this reference

### SCOPE OF THE INVENTION

This invention pertains to the treatment of the upper airway in a mammalian body and, more particularly, to the treatment of soft tissue in the roof of the mouth for reducing upper airway obstructions.

### BACKGROUND

Primary snoring results from the vibration of the soft palate and uvula caused by breathing during sleep. Snoring is not only disruptive to sleep and family life, it may also serve as a precursor to hypertension, heart trouble and stroke. It may also be a symptom of obstructive sleep apnea, where the obstruction is total and thus results in the cessation of breathing, sometimes for many seconds.

There are known surgical procedures for treating snoring. In a laser-assisted uvula palatoplasty (LAUP) procedure, a laser is used to ablate or vaporize the uvula and portions of the soft palate. In a uvulopalatopharyngoplasty (UPPP) procedure, the palate is folded over on itself and stitched into place thus surgically trimming and tightening the throat tissue and creating stiffening scar tissues. In a radio frequency ablation (RFA) procedure, a custom electrode is used to deliver radio frequency energy to the soft palate to stiffen the soft palate. These procedures can all involve post-operative pain and complications including bleeding and airway obstruction. In an injection snoreplasty procedure, a biocompatible material such as sodium teradecyl sulphate (Sotradecol), a sclerosing agent typically used to treat varicose veins, is injected into the soft palate to create scar tissues and to stiffen the soft palate and prevent fluttering. Issues have been raised concerning the safety of Sotradecol for injection into the soft palate, as it is recommended that Sotradecol is for intravenous use only. In addition, there are possible side effects if the agent is injected non-intravenously. These include severe local effects such as tissue necrosis, sloughing and necrosis after extravasation, and allergic reactions including anaphylaxis.

Accordingly, there is a need for an improved method and apparatus for treating upper airway obstructions that are not only non-invasive and less painful, but also safe and economical.

### SUMMARY

A method is provided for treating an airway in a mammalian body having a mouth, soft palate, uvula, throat and tongue. An implant-forming material is introduced into at least one of the soft palate and uvula for treating at least one of snoring and sleep apnea. An apparatus and kit are provided.

### BRIEF DESCRIPTION OF THE DRAWINGS

FIG. 1 is a plan view of one embodiment of a kit of the invention, somewhat schematic and partially cut away, for treating the upper airway in a mammalian body and including an apparatus of the invention.

FIG. 2 is a cross-sectional view of one embodiment of the apparatus of the kit of FIG. 1 taken along the line 2-2 of FIG. 1.

FIG. 3 is a cross-sectional view of the embodiment of the apparatus of FIG. 2 taken along the line 3-3 of FIG. 2.

FIG. 4 is a front view of an interior of a mouth of a mammalian body as treated by the formation of a plurality of implants in accordance with a first method of the present invention.

FIG. 5 is a front view of an interior of a mouth of a mammalian body as treated by the formation of a plurality of implants in accordance with a second method of the present invention.

FIG. 6 is a schematic side view of an interior of a mouth being treated with the embodiment of the apparatus of FIG. 2 in accordance with a step of one method of the present invention.

FIG. 7 is a schematic side view of an interior of a mouth being treated with the embodiment of the apparatus of FIG. 2 in accordance with a subsequent step of one method of the present invention.

FIG. 8 is a schematic view, similar to FIG. 7, of the apparatus of FIG. 2 coupled to a positive pressure device.

### DESCRIPTION OF THE INVENTION

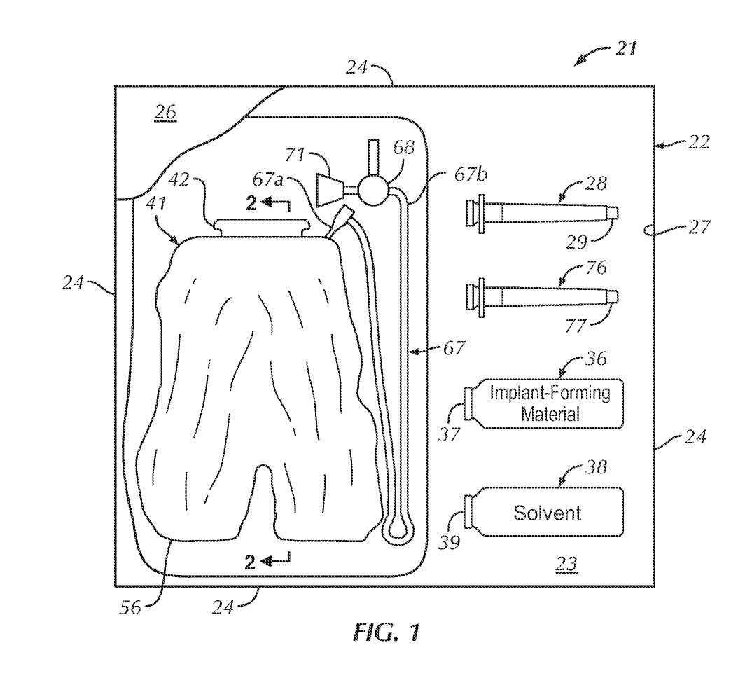

Referring to FIGS. 1-7, various embodiments of the invention are now described. It should be noted that the figures are only intended to facilitate the description of specific embodiments of the invention. They are not intended as an exhaustive description of the invention or as a limitation on the scope of the invention. In addition, an aspect described in conjunction with a particular embodiment of the present invention is not necessarily limited to that embodiment and can be practiced in any other embodiments of the present invention.

One embodiment of the invention, an apparatus and kit 21 for use in treating an airway, and in one embodiment an airway obstruction, in a mammalian body is provided and shown in FIG. 1. The kit 21 can include a package 22 made from any suitable material such as cardboard, paper or plastic for carrying the contents thereof. Exemplary package 22, as shown in FIG. 1, is a box that can be formed from a bottom wall 23, four sidewalls 24 and a top wall 26. A portion of the top wall 26 is cut away in FIG. 1 to reveal an internal space 27 formed by walls 23, 24 and 26. The contents of receptacle or package 22 are disposed in internal space 27.

Kit 21 may include a device for introducing an implant-forming material into at least one of the soft palate and uvula of the mammalian body. Such device can include a syringe 28 having a Luer-fitting portion 29. A container or vial 36 of any suitable implant forming material can be further included within kit 21. The container 36 may have a cap 37 that can be removably coupled to luer-fitting portion 29 of the syringe 28.

The implant-forming material is at least one material. In one embodiment, the implant-forming material may be any suitable material from which an implant can be formed when the material, separately or in conjunction with another material, is introduced into the tissue of the mammalian body. Although aqueous or non-aqueous solutions are amongst the fluids that can be used, in one embodiment an inert, nonresorbable material is used. One such material comprises at least one solution which when introduced into the body forms a nonbiodegradable solid. As used herein, a solid means any substance that does not flow perceptibly under moderate stress, has a definite capacity for resisting forces which tend to deform it (such as compression, tension and strain) and under ordinary conditions retains a definite size and shape; such a solid includes, without limitation, spongy and/or porous substances. One such embodiment of the at least one solution is first and second solutions which when combined in the body form the nonbiodegradable solid. Another such embodiment is a nonaqueous solution which can be introduced into the body as a liquid and from which a solid thereafter precipitates. Such nonaqueous solution can be a solution of a biocompatible polymer and a biocompatible solvent which can optionally include a contrast agent for facilitating visualization of the solution in the body.

The implant-forming or bulking solution of the invention can be a composition comprising from about 2.5 to about 8.0 weight percent of a biocompatible polymer, from about 52 to about 87.5 weight percent of a biocompatible solvent and optionally from about 10 to about 40 weight percent of a biocompatible contrast agent having a preferred average particle size of about 10 microns or less. It should be appreciated that any percentages stated herein which include a contrast agent can be proportionally adjusted when the contrast agent is not utilized. Any contrast agent can be a water insoluble biocompatible contrast agent. The weight percent of the polymer, contrast agent and biocompatible solvent may be based on the total weight of the complete composition. In one embodiment, the water insoluble, biocompatible contrast agent may be selected from the group consisting of barium sulfate, tantalum powder and tantalum oxide. The biocompatible solvent can be dimethylsulfoxide (DMSO), ethanol, ethyl lactate or acetone.

The term “biocompatible polymer” refers to polymers which, in the amounts employed, are non-toxic, chemically inert, and substantially non-immunogenic when used internally in the patient and which are substantially insoluble in physiologic liquids. Suitable biocompatible polymers include, by way of example, cellulose acetates (including cellulose diacetate), ethylene vinyl alcohol copolymers, hydrogels (e.g., acrylics), poly(C1-C6) acrylates, acrylate copolymers, polyalkyl alkacrylates wherein the alkyl and alk groups independently contain one to six carbon atoms, polyacrylonitrile, polyvinylacetate, cellulose acetate butyrate, nitrocellulose, copolymers of urethane/carbonate, copolymers of styrene/maleic acid, and mixtures thereof. Copolymers of urethane/carbonate include polycarbonates that are diol terminated which are then reacted with a diisocyanate such as methylene bisphenyl diisocyanate to provide for the urethane/carbonate copolymers. Likewise, copolymers of styrene/maleic acid refer to copolymers having a ratio of styrene to maleic acid of from about 7:3 to about 3:7. The biocompatible polymer can also be non-inflammatory when employed in situ. The particular biocompatible polymer employed is not critical and can be selected relative to the viscosity of the resulting polymer solution, the solubility of the biocompatible polymer in the biocompatible solvent, and the like. Such factors are well within the skill of the art.

The polymers of polyacrylonitrile, polyvinylacetate, poly(C1-C6) acrylates, acrylate copolymers, polyalkyl alkacrylates wherein the alkyl and alk groups independently contain one to six carbon atoms, cellulose acetate butyrate, nitrocellulose, copolymers of urethane/carbonate, copolymers of styrene/maleic acid and mixtures thereof typically have a molecular weight of at least about 50,000 and preferably from about 75,000 to about 300,000.

Biocompatible polymers include cellulose diacetate and ethylene vinyl alcohol copolymer. In one embodiment, the cellulose diacetate has an acetyl content of from about 31 to about 40 weight percent. Cellulose diacetate polymers are either commercially available or can be prepared by art recognized procedures. The number average molecular weight, as determined by gel permeation chromatography, of the cellulose diacetate composition can be from about 25,000 to about 100,000, preferably from about 50,000 to about 75,000 and more preferably from about 58,000 to 64,000. The weight average molecular weight of the cellulose diacetate composition, as determined by gel permeation chromatography, can be from about 50,000 to 200,000 and preferably from about 100,000 to about 180,000. As is apparent to one skilled in the art, with all other factors being equal, cellulose diacetate polymers having a lower molecular weight impart a lower viscosity to the composition as compared to higher molecular weight polymers. Accordingly, adjustment of the viscosity of the composition can be readily achieved by mere adjustment of the molecular weight of the polymer composition.

Ethylene vinyl alcohol copolymers comprise residues of both ethylene and vinyl alcohol monomers. Small amounts (e.g., less than 5 mole percent) of additional monomers can be included in the polymer structure or grafted thereon provided such additional monomers do not alter the implanting properties of the composition. Such additional monomers include, by way of example only, maleic anhydride, styrene, propylene, acrylic acid, vinyl acetate and the like.

Ethylene vinyl alcohol copolymers are either commercially available or can be prepared by art recognized procedures. In one embodiment, the ethylene vinyl alcohol copolymer composition can be selected such that a solution of 8 weight-volume percent of the ethylene vinyl alcohol copolymer in DMSO has a viscosity equal to or less than 60 centipoise at 20° C. and preferably 40 centipoise or less at 20° C. As is apparent to one skilled in the art, with all other factors being equal, copolymers having a lower molecular weight will impart a lower viscosity to the composition as compared to higher molecular weight copolymers. Accordingly, adjustment of the viscosity of the composition as necessary for catheter delivery can be readily achieved by mere adjustment of the molecular weight of the copolymer composition.

As is also apparent, the ratio of ethylene to vinyl alcohol in the copolymer can affect the overall hydrophobicity/hydrophilicity of the composition which, in turn, can affect the relative water solubility/insolubility of the composition as well as the rate of precipitation of the copolymer in an aqueous solution. In one embodiment, the copolymers employed herein comprise a mole percent of ethylene of from about 25 to about 60 and a mole percent of vinyl alcohol of from about 40 to about 75, preferably a mole percent of ethylene of from about 40 to about 60 and a mole percent of vinyl alcohol of from about 40 to about 60.

The term “contrast agent” refers to a biocompatible (non-toxic) radiopaque material capable of being monitored during injection into a mammalian subject by, for example, radiography. The contrast agent can be either water soluble or water insoluble. Examples of water soluble contrast agents include metrizamide, iopamidol, iothalamate sodium, iodomide sodium, and meglumine. The term “water insoluble contrast agent” refers to contrast agents which are insoluble in water (i.e., has a water solubility of less than 0.01 milligrams per milliliter at 20° C.) and include tantalum, tantalum oxide and barium sulfate, each of which is commercially available in the proper form for in vivo use and preferably having a particle size of 10 μm or less. Other water insoluble contrast agents include gold, tungsten and platinum powders. Methods for preparing such water insoluble biocompatible contrast agents having an average particle size of about 10 μm or less are described below. In one embodiment, the contrast agent is water insoluble (i.e., has a water solubility of less than 0.01 mg/ml at 20° C.).

The term “biocompatible solvent” refers to an organic material liquid at least at body temperature of the mammal in which the biocompatible polymer is soluble and, in the amounts used, is substantially non-toxic. Suitable biocompatible solvents include, by way of example, dimethylsulfoxide, analogues/homologues of dimethylsulfoxide, ethanol, ethyl lactate, acetone, and the like. Aqueous mixtures with the biocompatible solvent can also be employed provided that the amount of water employed is sufficiently small that the dissolved polymer precipitates upon injection into a human body. In one embodiment, the biocompatible solvent is ethyl lactate or dimethylsulfoxide.

The term “encapsulation” as used relative to the contrast agent being encapsulated in the precipitate is not meant to infer any physical entrapment of the contrast agent within the precipitate much as a capsule encapsulates a medicament. Rather, this term is used to mean that an integral coherent precipitate forms which does not separate into individual components, for example into a copolymer component and a contrast agent component.

The compositions employed in the methods of this invention may be prepared by conventional methods whereby each of the components is added and the resulting composition mixed together until the overall composition is substantially homogeneous. For example, sufficient amounts of the selected polymer are added to the biocompatible solvent to achieve the effective concentration for the complete composition. In one embodiment, the composition comprises from about 2.5 to about 8.0 weight percent of the polymer based on the total weight of the composition and preferably from about 4 to about 5.2 weight percent. If necessary, gentle heating and stirring can be used to effect dissolution of the polymer into the biocompatible solvent, e.g., 12 hours at 50° C.

Sufficient amounts of the contrast agent can be then optionally added to the biocompatible solvent to achieve the effective concentration for the complete composition. In one embodiment, the composition comprises from about 10 to about 40 weight percent of the contrast agent and preferably from about 20 to about 40 weight percent and more preferably about 30 to about 35 weight percent. When the contrast agent is not soluble in the biocompatible solvent, stirring can be employed to effect homogeneity of the resulting suspension. In order to enhance formation of the suspension, the particle size of the contrast agent can be maintained at about 10 μm or less and in one embodiment at from about 1 to about 5 μm (e.g., an average size of about 2 μm). The appropriate particle size of the contrast agent can be prepared, for example, by fractionation. In such an embodiment, a water insoluble contrast agent such as tantalum having an average particle size of less than about 20 microns can be added to an organic liquid such as ethanol (absolute) preferably in a clean environment. Agitation of the resulting suspension followed by settling for approximately 40 seconds generally permits the larger particles to settle faster. Removal of the upper portion of the organic liquid followed by separation of the liquid from the particles generally results in a reduction of the particle size which is confirmed under an optical microscope. The process can be repeated until a desired average particle size is reached.

The particular order of addition of components to the biocompatible solvent is not critical and stirring of the resulting suspension is conducted as necessary to achieve homogeneity of the composition. Mixing/stirring of the composition may be conducted under an anhydrous atmosphere at ambient pressure. The resulting composition may be heat sterilized and can then be stored in sealed amber bottles or vials until needed.

Each of the polymers recited herein is commercially available but can also be prepared by methods well known in the art. For example, polymers are typically prepared by conventional techniques such as radical, thermal, UV, gamma irradiation, or electron beam induced polymerization employing, as necessary, a polymerization catalyst or polymerization initiator to provide for the polymer composition. The specific manner of polymerization is not critical and the polymerization techniques employed do not form a part of this invention. In order to maintain solubility in the biocompatible solvent, in one embodiment the polymers described herein are not cross-linked.

In another embodiment of the implant-forming or augmenting solution, the biocompatible polymer composition can be replaced with a biocompatible prepolymer composition containing a biocompatible prepolymer. In this embodiment, the composition comprises a biocompatible prepolymer, an optional biocompatible water insoluble contrast agent having an average particle size of about 10 μm or less and, optionally, a biocompatible solvent.

The term “biocompatible prepolymer” refers to materials which polymerize in situ to form a polymer and which, in the amounts employed, are non-toxic, chemically inert, and substantially non-immunogenic when used internally in the patient and which are substantially insoluble in physiologic liquids. Such a composition can be introduced into the body as a mixture of reactive chemicals and thereafter forms a biocompatible polymer within the body. Suitable biocompatible prepolymers include, by way of example, cyanoacrylates, hydroxyethyl methacrylate, silicon prepolymers, and the like. The prepolymer can either be a monomer or a reactive oligomer. In one embodiment, the biocompatible prepolymer is non-inflammatory when employed in situ.

Prepolymer compositions can be prepared by adding sufficient amounts of the optional contrast agent to the solution (e.g., liquid prepolymer) to achieve the effective concentration for the complete polymer composition. The prepolymer composition may comprise from about 10 to about 40 weight percent of the contrast agent and preferably from about 20 to about 40 weight percent and more preferably about 30 weight percent. When the contrast agent is not soluble in the biocompatible prepolymer composition, stirring can be employed to effect homogeneity of the resulting suspension. In order to enhance formation of the suspension, the particle size of the contrast agent can be maintained at about 10 μm or less and more preferably at from about 1 to about 5 μm (e.g., an average size of about 2 μm).

When the prepolymer is liquid (as in the case of polyurethanes), the use of a biocompatible solvent is not absolutely necessary but may be utilized to provide for an appropriate viscosity in the implant-forming solution. In one embodiment, when employed, the biocompatible solvent may comprise from about 10 to about 50 weight percent of the biocompatible prepolymer composition based on the total weight of the prepolymer composition. When a biocompatible solvent is employed, the prepolymeric composition typically comprises from about 90 to about 50 weight percent of the prepolymer based on the total weight of the composition.

In one embodiment, the prepolymer comprises cyanoacrylate which can be employed in the absence of a biocompatible solvent. When so employed, the cyanoacrylate adhesive can be selected to have a viscosity of from about 5 to about 20 centipoise at 20° C.

The particular order of addition of components is not critical and stirring of the resulting suspension is conducted as necessary to achieve homogeneity of the composition. Mixing/stirring of the composition can be conducted under an anhydrous atmosphere at ambient pressure. The resulting composition can be sterilized and then stored in sealed amber bottles or vials until needed.

Other suitable implant-forming materials for introduction into one or both of muscle layers 201 and 202 include injectable bioglass as described in Walker et al., “Injectable Bioglass as a Potential Substitute for Injectable Polytetrafluorethylene Particles”, J. Urol., 148:645-7, 1992, small particle species such as Polytetrafluorethylene (PTFE) particles in glycerine such as Polytef®, biocompatible compositions comprising discrete, polymeric and silicone rubber bodies such as described in U.S. Pat. Nos. 5,007,940, 5,158,573 and 5,116,387 to Berg, biocompatible compositions comprising carbon coated beads such as disclosed in U.S. Pat. No. 5,451,406 to Lawin, collagen and other biodegradable material of the type disclosed in U.S. Pat. No. 4,803,075 to Wallace et al. and other known injectable materials.

Specific embodiments of implant-forming solutions suitable for use in the apparatus and methods of the invention are described in U.S. Pat. No. 5,667,767 dated Sept. 16, 1997, U.S. Pat. No. 5,580,568 dated Dec. 3, 1996 and U.S. Pat. No. 5,695,480 dated Dec. 9, 1997 and International Publication Number WO 97/45131 having an International Publication Date of Dec. 4, 1997, the entire contents of which are incorporated herein by this reference.

Kit 21 further optionally can include a container such as vial 38 of a biocompatible solvent such as DMSO. The vial 38 includes a cap 39 that can be removably coupleable to a luer-fitting portion of a syringe (not shown).

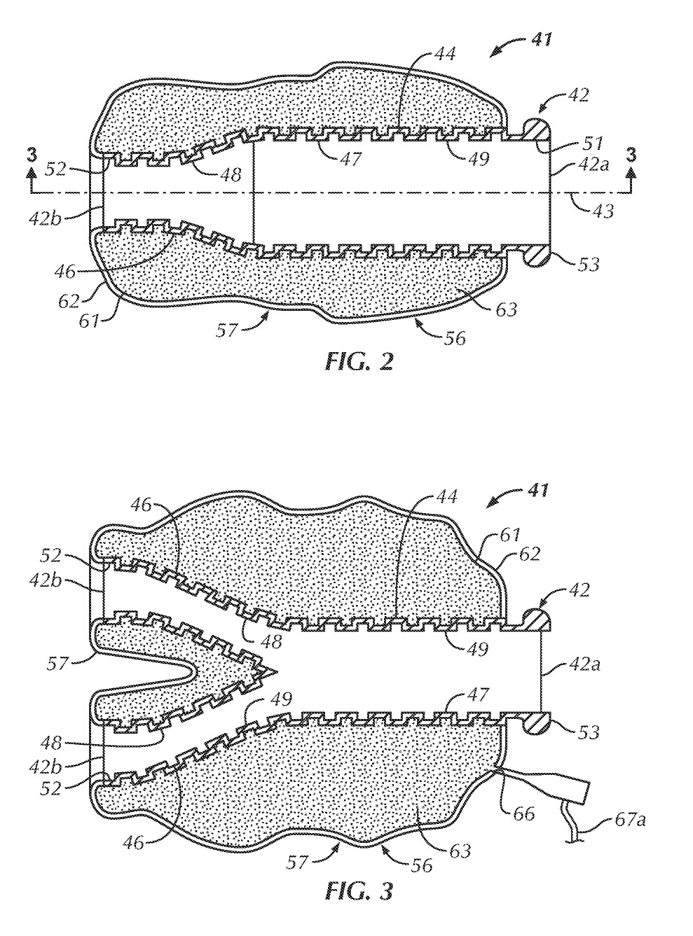

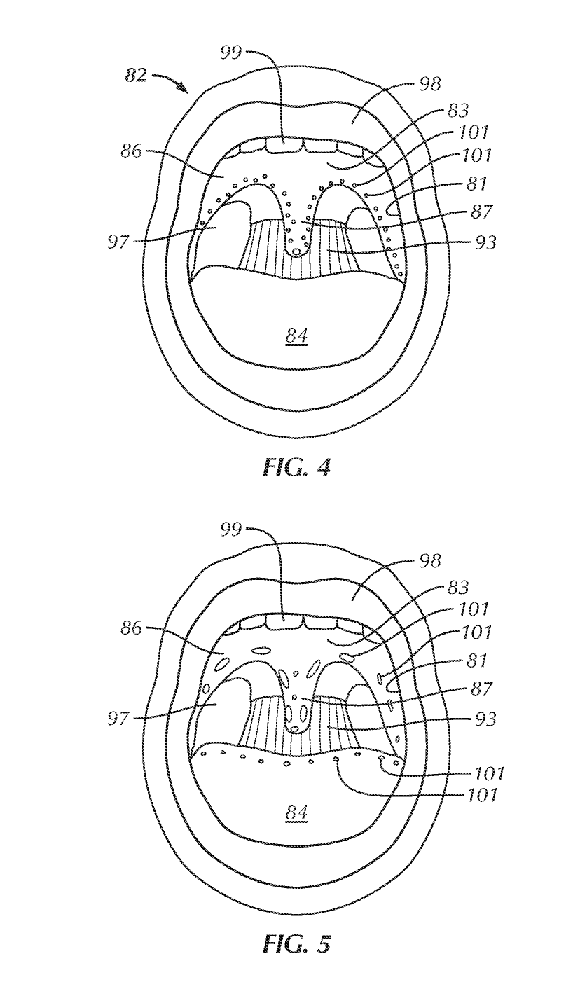

An apparatus 41 typically for use in molding the upper wall of a mouth of a mammalian body can be included in kit 21 and, as more specially shown in FIGS. 2 and 3, includes an inner portion or central member 42 having an anterior portion 42a and a posterior portion 42b and a longitudinal axis 43 extending between the portions 42a and 42b. In one embodiment, the member 42 can have a length to permit the anterior portion 42a of the member to be placed in the vicinity of the opening of the mouth 81 of a patient and the posterior portion 42b to be placed in the vicinity of the uvula 87 of the patient and for example in the vicinity of the opening of the throat 93 of the patient. The member 42 can have a shape of a “Y” and, in this regard, can have a base portion or trunk 44 extending from the anterior portion 42a and first and second arm portions or branches 46 extending posterially and transversely of longitudinally axis 43 at posterior portion 42b. Base portion 44 can have a length to permit the anterior portion of the trunk 44 to be placed in the vicinity of the opening of the mouth 81 of the patient and the posterior portion of the trunk 44 to be placed forward of the uvula 87 of the patient and in one embodiment has a length of approximately ten centimeters. Each of the arm portions 46 can have a length to permit the arm portion to extend from the posterior portion of the trunk 44 beyond the uvula 87 and in one embodiment into the vicinity of the opening of the throat 93 of the patient. The arm portions can extend outwardly at an acute angle from longitudinal axis 43. A passageway 47, that can have a diameter in one embodiment ranging from 1.5 to 2.0 centimeters and in another embodiment of approximately 1.5 centimeters, can extend through the base portion 42. First and second smaller passageways 48, that can each have a diameter ranging from 0.5 to 1.0 centimeter, can extend respectively through first and second arm portions 46. In general, the measurements set forth above are exemplary only. The apparatus or tubular member 42 is sized and shaped to fit within the mouth, throat or nose of any suitable patient, it being understood that different sized apparatus can be provided for different sized patients. In this regard, the apparatus 42 can be sized to fit within the mouth, throat or nose of an adult, a child or both. Generally, the tubular member 42 can be made from any suitable materials such as metals, plastics and elastomers with or without reinforcers added thereto. For example, the tubular member can be made from a plastic material that is bendable so as to be capable of assuming a plurality of set shapes. The tubular member may also comprise, for example, a reinforced material such as silicone reinforced with plastic or metal braids or strip. In this regard, tubular wall 49 forming passageways 47 and 48 of member 42 may be formed from a plurality of longitudinally spaced-apart serrations so as to facilitate the bending of the member 42 relative to longitudinal axis 43. Passageway 47 may comprise an opening 51 at anterior portion 42a and first and second passageways 48 have respective openings 52 at posterior portion 42b. A flange 53 can be provided on the anterior portion 42a of the tubular member 42 at opening 51.

Tubular apparatus or mouthpiece 41 may include an outer portion or annular pouch 56 having an inner wall formed by tubular member 42 and an outer wall 57 outside of the tubular member 42. The flexible outer wall of hermetically-sealed pouch 56 may have one end secured to the anterior portion 42a and an opposite end secured to posterior portion 42b. The pouch includes an interior portion 58 extending between first and second arm portions 46 (see FIG. 3). The outer wall 57 can be made from any suitable flexible material such as polyethylene, polyurethane or polyvinylchloride and can be formed from an inner membrane 61 and an outer membrane 62 so as to be doubled walled and thus less likely to puncture. The posterior end of tubular member 42 can be made from any suitable material such as polyester or woven polyester fibers and can be less rigid and/or softer or lighter than the remainder of the tubular member 42 so as to make the arm portions 46 flexible and thus capable of moving during placement of the mouthpiece into the mouth of the patient. A suitable moldable material 63, such as alginate plus water, can be disposed within pouch 56. The moldable material can also be a suitable silicone, such as dental silicone or silicone putty like OPTOSIL silicone putty sold by Heraeus Kulzer Inc. of Armonk, N.Y., and may be enhanced with an imageable agent, for example a contrast agent that is visible under fluoroscopy. Pouch 56 can be coated with any suitable medicament such as a sedating product.

At least one port 66 can be provided in pouch 56 for permitting the introduction of an agent for causing the moldable material 63 to change from a malleable state to a hardened state (see FIG. 3). The port 66 can be located in the anterior portion of the mouthpiece 41, and thus near the mouth of the patient when the mouthpiece is disposed in the mouth of the patient, so as to permit the agent to be introduced into the pouch 56 when the mouthpiece is placed in the mouth of the patient. At least one tube 67, as shown in FIGS. 1 and 3, may be included in mouthpiece 41 and can have a first end 67a permanently or removably coupled to port 66 and a second end 67b that can be secured to a valve 68 coupled to any suitable connector such as a luer-fitting 71. The mouthpiece 41 can be provided with a plurality of ports 66 located in spaced apart positions on the pouch 56 to facilitate distribution of the agent within the pouch. When multiple ports 66 are so provided, the tube 67 can have multiple ends for respectively coupling to such ports and/or a plurality of tubes 67 can be provided. Mouthpiece 41 can optionally be hermetically sealed within a container 72 made from any suitable material such as plastic, and tube 67 and fitting 71 can be further included within the container 72 , in each case for enhancing sterility of these kit components.

It is appreciated that moldable material 63 can be of a type that does not require the introduction of an agent for causing the moldable material to change from a malleable state to a hardened state. In such an embodiment, a port 66 would not be required in pouch 66. Any other suitable moldable material that can change from a malleable state to a hardened state can be provided. In this regard, certain moldable materials 63 may not require that mouthpiece 41 be provided with an outer wall 57. It is appreciated that other embodiments of apparatus or mouthpiece of the present invention can be provided.

Kit 21 can further include a second syringe 76 having a luer-fitting portion 77 that can be removably coupled to luer-fitting 71.

In one method of the invention, kit 21 can be utilized for forming a plurality of implants in the mouth 81 of a mammalian body. A portion of a mammalian body 82, more specifically the oral cavity or mouth 81, is shown in FIGS. 4-7. The hard palate 83 overlies the tongue 84 and forms the roof of the mouth. The hard palate includes a bone support and does not materially deform during breathing. The soft palate 86 is soft and is made up of mucous membrane, fibrous and muscle tissue extending rearward from the hard palate. A trailing end of the soft palate is the uvula 87. Since the soft palate is not structurally supported by bone or hard cartilage, the soft palate droops down from the plane of the hard palate. The pharyngeal airway 88 passes air from the mouth 81 and the nasal passages 89 of the nose into the trachea 91. The epiglottis 92, a lid-like cartilaginous structure, overhangs the entrance to the trachea. The portion of the pharyngeal airway defined between opposing surfaces of the upper surface of the soft palate and the wall of the throat 93 is the nasopharynx 94. The throat extends downwardly into the esophagus 96. The tonsils 94 border the opening between the mouth 81 and the throat 93, and the lips 98 and teeth 99 define the opening to mouth 81.

During normal breathing, the soft palate 86 and the uvula 87 are in a relaxed state with the nasopharynx 94 unobstructed so that air can flow freely into the trachea 91 from both the mouth 81 and the nasal passages 91. During swallowing, the uvula flexes and extends to close the nasopharynx 94 thereby preventing fluid flow from the mouth 81 to the nasal passages 89. Simultaneously, the epiglottis 92 closes the trachea 91 so that food and drink pass only into the esophagus 96 and not the trachea. The uvula 87 is a valve to prevent regurgitation of food into the nasal passages 89 and the nose. The uvula also regulates airflow through the nasal passages while talking.

Snoring can be caused by one or both of the soft palate 86 and the uvula 87 flapping back and forth against the tongue 84 or more likely the back of the throat 93. For example, the uvula may be too long or too floppy. For example, during the breathing cycle the soft palate 86 and the uvula 87 can be sucked into the nasopharynx 94 obstructing such airway in a repeating cycle. When the area at the back of the throat 93 narrows, the same amount of air passing through this smaller opening can cause the tissues surrounding the opening to vibrate, which in turn can cause the sounds of snoring.

The patient can be sedated locally prior to the formation of implants in the oral cavity. Syringe 28 is coupled to cap 37 of vial 36 and filled with the implant-forming material within the vial 36. A needle (not shown) can be then coupled to syringe 28 and extended into the mouth and tissue of the soft palate and/or uvula to create one or more implants 101 in such tissue. The quantity of implant-forming material in one embodiment can range from 0.075 to 0.200 cubic centimeters per implant, in another embodiment can range from 0.08 to 0.15 cubic centimeters per implant and in yet another embodiment can range from 0.07 to 0.10 cubic centimeters per implant. When the implant-forming material is a biocompatible polymer in a biocompatible solution of the type discussed above, the biocompatible polymer precipitates once placed in the tissue to form one or more discrete deposits or solid implants. Such implants are nonbiodegradable. A suitable biocompatible solvent, such as the solvent within vial 38, can be utilized during the procedure, for example to cleanse or prime the needle utilized for introducing the implant-forming material into the tissue of the mouth. The contrast agent with the implant-forming material facilitates visualization of such material within body 82.

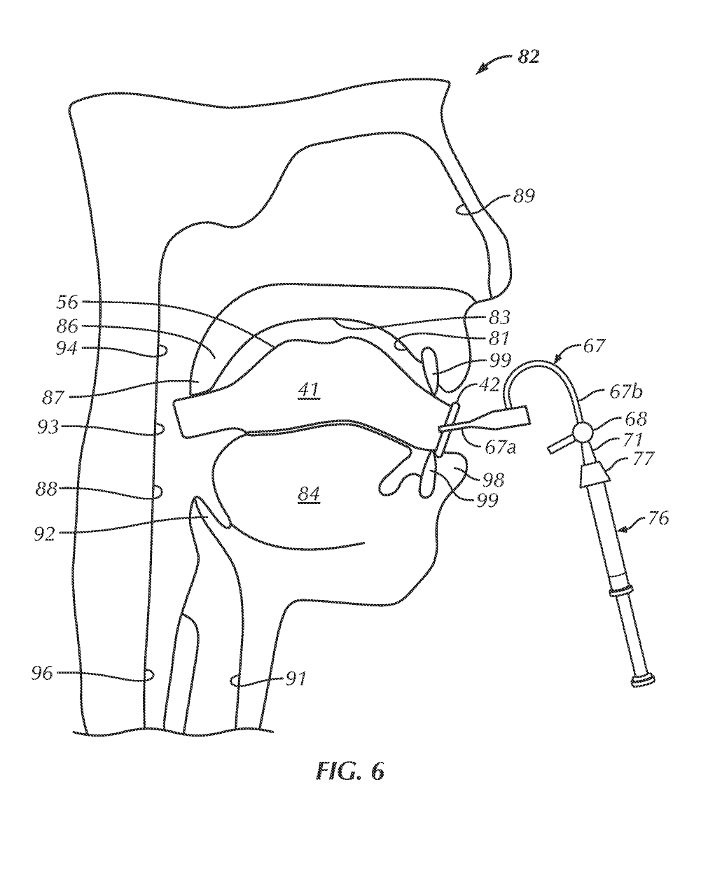

Any suitable arrangement or configuration of implants can be provided. In this regard, the number, size and configuration of implants formed in the soft palate 86, the uvula 87 or the soft palate and the uvula can vary. In one embodiment, as shown in FIG. 4, a plurality of spaced-apart implants 101 can be created along the periphery of both the soft palate 86 and uvula 87. The implants 101 can be small and circular in shape as shown in FIG. 4, elongate or spherical in shape as shown in FIG. 5 or any combination of the foregoing. Implants 101 can further be formed in tongue 84, as shown in FIG. 5. In one embodiment, the implants 101 can be formed in either the submucosal or muscle layers of the soft palate, uvula and tongue. The implants can be formed in a symmetrical arrangement relative to the uvula. The implants 101 are preferably formed in tissue away from nerves, blood vessels and arteries. The implants 101 reduce the distensibility of the tissue in which they have been formed, for example the tissue of the soft palate 86, uvula 87 and/or tongue 84, so as to increase the tone and rigidity of such structures and thus minimize the undesirable flapping or vibration of such structures, or closure of airways by such members, when the patient is breathing.

After the formation of the implants in one or all of the soft palate 86, uvula 87 and tongue 84, mouthpiece 41 can be placed into the mouth 81 for facilitating molding of the mouth and thus further aiding in the reduction of snoring or sleep apnea. In general, mouthpiece 41 is sized and shaped complementary to the oral cavity of the patient. Different sizes of the mouthpiece 41 can be provided so as to accommodate different sized mouths. The posterior portion 42b of the mouthpiece is introduced into the mouth and moved to the rear of the mouth so that first and second arm portions 46 are disposed on each side of the uvula 87. In this manner, the uvula depends between the arm portions 46 of the mouthpiece 41. The flexibility of the posterior portion 42b of the tubular member 42 permits the first and second arm portions 46 to move during placement of the mouthpiece, for example to accommodate the uvula 87. Once mouthpiece 41 has been disposed within the mouth 81, luer-fitting portion 77 of the second syringe or injector 76 can be coupled to luer-fitting 71 to introduce a suitable liquid, such as water or saline solution, into pouch 56 so as interact with or complete the formation of the moldable material 63 within the pouch. The introduction of such liquid into the pouch 56 increases the volume of the pouch and thus further serves to inflate the pouch. It is appreciated that the water or other solution can be introduced into pouch 56 of mouthpiece 41 before placement of the pouch in the mouth 81.

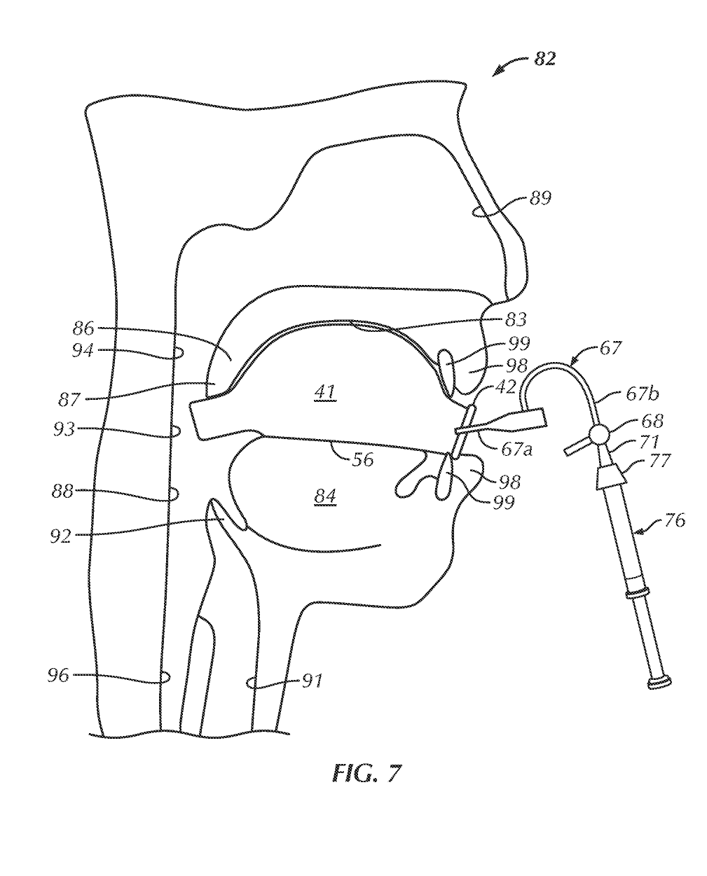

Mouth 81 is then closed by the patient and held in such closed position for an appropriate period of time to mold the pouch into the shape of the patient's mouth 81, particularly the shape of the soft palate 86 and uvula 87. The bendable material of tubular member 42 facilitates changes in the shape of the mouthpiece 41 so that the mouthpiece can more easily assume the shape of the mouth. In this regard, the tubular member can bend during molding. The patient's teeth can be clamped to the tubular member 42 adjacent flange 53, as illustrated in FIGS. 6 and 7. Optional passageways 47 and 48 permit patient breathing through mouth 81 while the mouthpiece 41 is disposed in the patient's mouth. In one procedure of the invention, the mouthpiece 41 can remain in the patient's mouth 81 for approximately five minutes to accomplish proper molding of the mouthpiece 41.

Once the moldable material 63 has hardened, causing the tubular member 42 to retain its changed shape, mouthpiece 41 can be used by the patient to facilitate rebuilding the soft palate and the uvula and thus limit the subsequent reduction of the volume and size of the patient's airways. In this regard, mouthpiece 41 can act as a memory device for retaining the shape of mouth 81 immediately following the creation of implants 101 in the mouth. For example, if implants 101 serve to cause an inflammatory reaction in the mouth tissue and a resulting increase of the volume of such tissue, mouthpiece 41 can serve to decrease muscle distensibility and increase yield pressure so that the tissues in which implants have been formed, such as the uvula and soft palate, remain tonic. In one rebuilding procedure of the invention, the molded mouthpiece 41 can be placed in the patient's mouth for a suitable period of time, for example for several hours, following formation of implants 101. The molded mouthpiece 41 can be placed in the patient's mouth 81 during sleep for a suitable number of days following the implant-forming procedure. The mouthpiece can be so utilized for a suitable period of time, for example for several hours following formation of implants 101. The mouthpiece is preferably cleaned after each use, for example by placement in a container of a suitable cleaning solution.

In another procedure of the invention, the mouthpiece 41 can be placed in mouth 81 of the patient such as illustrated in FIG. 6 at an appropriate time, inflated and hardened to form a mold of the shape of the patient's mouth, such as illustrated in FIG. 7, and then removed from the mouth 81 before the formation of implants 101 in the tissue of the mouth. After the formation of implants 101, the mouthpiece is reintroduced into the mouth 81 at suitable times to facilitate shaping or rebuilding of the tissue in the mouth in a desired manner. For example, the mouthpiece can be placed into the mouth to maintain a desired shape of the mouth until the moldable material 63 of the, mouthpiece 41 sets. In a further procedure of the invention, adequate measurements of the interior of the mouth 81 are taken, for example by instruments introduced into the mouth, pictures, radiological techniques or otherwise, to permit the construction of mouthpiece 41 or a similar mold of the mouth, for example the upper rear of the mouth, prior to the formation of implants 101 in the tissue of the mouth. After the formation of implants 101, such mouthpiece or similar mold is reintroduced into the mouth 81 at suitable times to facilitate shaping or rebuilding of the tissue in the mouth, for example in the uvula and soft palate, in a desired manner such as any of the procedures discussed above.

It is appreciated that other embodiments of the mouthpiece can be provided in which one or more lumens are provided in the tubular member 42 for facilitating the introduction and distribution of the agent within the pouch 56 to interact with the moldable material 63 in the pouch. When a plurality of lumens are provided, the lumens can be circumferentially spaced apart around the tubular member and can extend longitudinally along the tubular member 42 for the same or different lengths. For example, one or more lumens may extend to the first arm portion 46 and one or more lumens may extend to the second arm portion 46, any multiple lumens in each arm portion being circumferentially and/or longitudinally spaced from each other. Additionally, one or more lumens may extend along base portion 44 and, where multiple lumens are provided, such lumens can be circumferentially and/or longitudinally spaced from each other. In one such embodiment, the one or more lumens communicate with one or more ports located for example in flange 53 at the anterior portion 42a of the tubular member 42.

In another embodiment and procedure of the invention, an apparatus or mold can be provided that has a size and shape similar to apparatus or mouthpiece 41 and an outer surface that is moldable or molded to have the shape of the inner surface of the patient's mouth before the formation of implants therein. In one embodiment, the apparatus or mold can be formed in the patient's mouth, by hand or otherwise, from a putty, for example putty or moldable materials of the type used in dental procedures. The apparatus of mold (not shown) can be provided with needle access points, target spots or marked locations such that when the apparatus or mold is placed in the mouth of the patient a needle of injectable or implant-forming material can be introduced through the apparatus or mold into the desired tissue of the mouth so as to form implants therein, for example in the manner discussed above. The needle access points or marked locations on the interior of the apparatus or mold can serve to guide the needle to the desired tissue of the mouth. In one embodiment, at least the outer wall of the apparatus or mold is rigid so as to facilitate proper placement of the injectable or implant-forming material in the patient's mouth. The apparatus or mold can serve to maintain proper or desired mouth shape until the injectable or implant-forming material sets, and if rigid can minimize undesirable bulging or swelling of the tissue of the mouth.

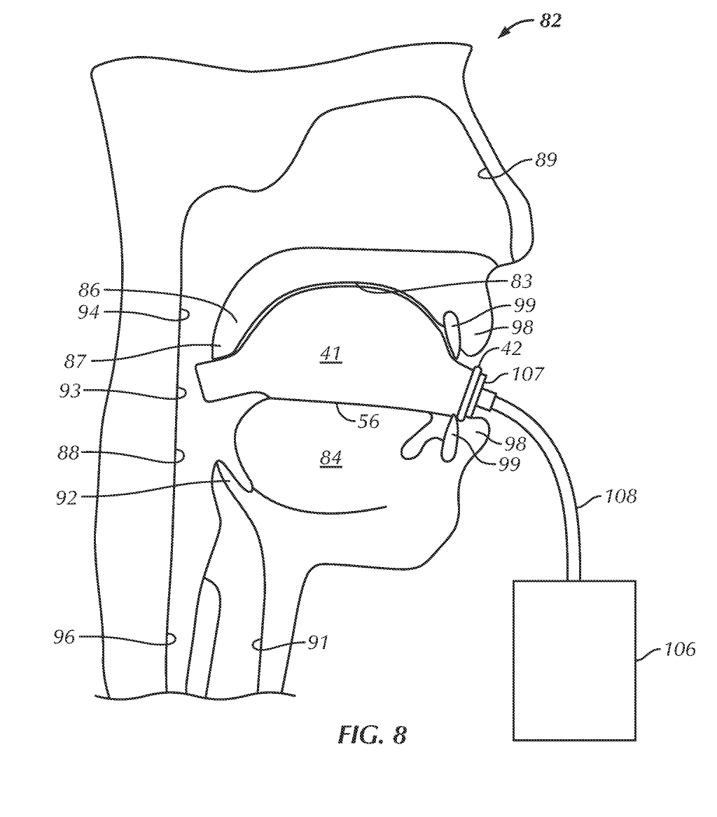

In an application where the patient is suffering from severe sleep apnea, after mouthpiece 41 has been introduced into the mouth 81 of the patient and the moldable material 63 fully formed in the manner discussed above, the mouthpiece can be coupled to any suitable positive pressure external air device 106 and used while the patient is asleep (see FIG. 8). In this regard, a suitable coupler 107 can be connected to anterior portion 42a of the tubular member 42 of mouthpiece 41 and a conventional tube 108 provided to extend between the coupler 107 and the device 106. The device 106 serves to supply positive pressure to passageways 47 and 48 of the mouthpiece 41 and thus to the mouth 81 and pharyngeal airway 88 of the patient. Although device 106 is shown in FIG. 8 as being a portable device, such as of the type manufactured by ResMed Corp in Poway, Calif., nonportable and devices with or without batteries can be utilized.

From the foregoing it will be appreciated that, although specific embodiments of the invention have been described herein for purposes of illustration, various modifications may be made without deviating from the spirit and scope of the invention.

What is claimed is:

1. A method of treating an airway in a mammalian body having a mouth, soft palate, uvula, throat and tongue comprising the steps of introducing an implant-forming material into at least one of the soft palate and uvula for treating at least one of snoring and sleep apnea.

2. The method of claim 1 wherein the introducing step includes introducing a solution into at least one of the soft palate, uvula, throat and tongue and forming a nonbiodegradable solid from the solution in at least one of the soft palate, uvula, throat and tongue.

3. The method of claim 2 wherein the solution includes a biocompatible polymer and a biocompatible solvent.

4. The method of claim 3 wherein the solution of the biocompatible polymer and the biocompatible solvent has a composition comprising from about 2.5 to about 8.0 weight percent of a biocompatible polymer, from about 10 to about 40 weight percent of a water insoluble biocompatible contrast agent and from about 52 to about 87.5 weight percent of a biocompatible solvent.

5. The method of claim 2 wherein the solution includes a contrast agent.

6. The method of claim 2 wherein the solution includes a prepolymer and wherein the forming step includes polymerizing the prepolymer in situ to form an implant in the wall.

7. The method of claim 2 wherein the forming step includes precipitating an implant from the solution.

8. The method of claim 1 wherein the uvula includes muscle fiber and the introducing step includes introducing the implant-forming material into the muscle fiber of the uvula.

9. A method of treating an airway in a mammalian body having a mouth, soft palate, uvula, throat and tongue comprising the steps of introducing an implant-forming material into at least one of the soft palate and uvula and inserting an apparatus into the mouth so as to be in contact with at least the soft palate and uvula to remold at least one of the soft palate and uvula and thus treat at least one of snoring and sleep apnea.

10. The method of claim 1 wherein the apparatus has an outer portion formed from a moldable material.

11. The method of claim 10 wherein the moldable material is silicone.

12. The method of claim 10 wherein the apparatus has an inner portion provided with a passageway for permitting the passage of air.

13. A method of treating an airway in a mammalian body having a mouth, soft palate, uvula, throat and tongue comprising the steps of forming a mold of at least one of the soft palate and uvula, introducing an implant-forming material into at least one of the soft palate and uvula and inserting the mold into the mouth in contact with at least the soft palate and uvula.

14. The method of claim 13 wherein the mold is provided with a passageway for permitting the passage of air through the mold when the mold is disposed in the mouth.

15. An apparatus for treating an airway in a mammalian body having a mouth formed by tissue that includes an uvula and a soft palate comprising an apparatus adapted for use with the mammalian body, the apparatus having a size for placement in the mouth and having an outer portion formed from a moldable material for creating a mold of at least one of the soft palate and uvula when the apparatus is placed in the mouth.

16. The apparatus of claim 15 wherein the moldable material is hardenable after being molded to a desired shape.

17. The apparatus of claim 15 wherein the apparatus has an inner portion that includes a tube for permitting the passage of air.

18. An apparatus for use in treating an airway in a mammalian body having a mouth formed by tissue that includes an uvula and a soft palate comprising a Y-shaped tubular member having a base portion and first and second arm portions extending and inclined from the base portion whereby the tubular member is adapted for placement in the mouth so that the uvula extends between the first and second arm portion, a passageway extending through the tubular member for permitting breathing through the tubular member and a moldable material extending around the tubular member for engaging the uvula and soft palate.

19. The apparatus of claim 18 wherein the tubular member is bendable.

20. The apparatus of claim 19 wherein the tubular member is made from a rigid material.

21. A kit for use in treating an airway in a mammalian body having a mouth formed by tissue that includes an uvula, soft palate, throat and tongue comprising a package, an apparatus adapted for use with the mammalian body provided within the package, the apparatus having a size for placement in the mouth and having an outer portion provided with a material that is moldable to the shape of at least one of the uvula and the soft palate, and a container of an implant-forming material carried by the package for introduction into at least one of the uvula and the soft palate.

22. The kit of claim 21 further comprising a container of a biocompatible solvent carried within the package.

23. The kit of claim 21 further comprising a syringe for introducing the implant-forming material into at least one of the uvula, soft palate, throat and tongue.

24. The kit of claim 21 wherein the implant-forming material is a nonaqueous solution for forming a nonbiodegradable solid in at least one of the uvula, soft palate, throat and tongue.

25. The kit of claim 21 wherein the apparatus has an inner portion that includes a tube for permitting air to pass through the apparatus.
